(19) United States
(12) Patent Application Publication
(10) Pub. No.: US 2025/0123237 A1
(43) Pub. Date: Apr. 17, 2025

(54) TRANSISTOR SENSOR FOR DISTINGUISHING CELL HETEROGENEITY AND METHOD FOR USING THE SAME

(71) Applicant: National Yang Ming Chiao Tung University, Hsinchu City (TW)

(72) Inventors: Tai-Ming Ko, Hsinchu City (TW); Yuh-Shyong Yang, Hsinchu City (TW); Kuan-Hsing Chen, Taichung City (TW); Che-Ming Lin, Zhubei City (TW)

(73) Assignee: National Yang Ming Chiao Tung University, Hsinchu City (TW)

(21) Appl. No.: 18/606,570

(22) Filed: Mar. 15, 2024

(30) Foreign Application Priority Data
Oct. 12, 2023 (TW) 112139035

(51) Int. Cl. G01N 27/414; B01L 3/00; G01N 33/68

(52) U.S. Cl. CPC G01N 27/4145; B01L 3/502715; G01N 33/6854

(57) ABSTRACT

A transistor includes a field effect transistor, a surface modification layer, and a cell detection layer. The field effect transistor includes a source region, a drain region, a channel region, a gate dielectric layer, and a gate. The drain region is spaced apart from the source region in a first direction. The channel extends in the first direction and is disposed between the source region and the drain region. The gate dielectric layer is disposed below the channel region. The gate is disposed below the gate dielectric layer. The surface modification layer is disposed on the channel region. The cell detection layer is disposed on the surface modification layer and includes a plurality of antibodies, wherein the antibodies are configured to identify cell surface antigens, and the cell detection layer is configured to capture cells identified by the antibodies.

### CROSS-REFERENCE TO RELATED APPLICATION

This application claims priority to Taiwan Application Serial Number 112139035, filed Oct. 12, 2023, which is herein incorporated by reference in its entirety.

### BACKGROUND

### Field of Invention

The present disclosure relates to a cell sensor and a detection method of using the cell sensor to distinguish heterogeneous cells.

### Description of Related Art

In the process of cell examination or cell therapy (such as immunotherapy for cancer), an important step is to identify heterogeneous cells. However, different types of cells are often mixed in a biological sample, such as tumor tissue. In conventional methods, identifying these heterogeneous cell types usually requires marking the cells with labels such as antibodies and using instruments like flow cytometry to identify the cell types in the sample according to the optical signals emitted from the labels. However, a larger quantity of reagents and consumables is required, leading to higher testing costs, and larger instruments are needed for the examination.

### SUMMARY

Some embodiments of the present disclosure provide a transistor sensor including a field effect transistor, a surface modification layer, and a cell detection layer. The field effect transistor includes a source region, a drain region, a semiconductor channel, a gate dielectric layer, and a gate. The drain region is spaced apart from the source region in the first direction. The semiconductor channel extends in the first direction and is disposed between the source region and the drain region. The gate dielectric layer is disposed under the semiconductor channel. The gate is disposed under the gate dielectric layer. The surface modification layer is disposed on the semiconductor channel. The cell detection layer is disposed on the surface modification layer and includes a plurality of antibodies, wherein the antibodies are configured to identify a cell surface antigen, and the cell detection layer is configured to capture cells identified by the antibodies.

The transistor sensor of the present disclosure can sense the cell type of interest by distinguishing the differences of cell surfaces. For example, antigens on the cell surface can be recognized by corresponding antibodies in the cell detection layer of the transistor sensor.

In some embodiments, the surface modification layer includes siloxane compounds.

In some embodiments, the cell surface antigen is a leukocyte differentiation antigen.

In some embodiments, the semiconductor channel has a length in the first direction to allow the cell detection layer to capture cells. In some embodiments, the length ranges from about 50 μm to 1000 μm.

In some embodiments, the surface modification layer is connected with the plurality of antibodies of the cell detection layer through a plurality of terminal aldehyde groups.

In some embodiments, the cells are white blood cells.

In some embodiments, the heterogeneous cells detected include different lymphocyte subtypes. The lymphocyte subtypes include but are not limited to B lymphocytes, T lymphocytes, NK cells, CD4 T lymphocytes, CD8 T lymphocytes, or the like.

In some embodiments, the plurality of antibodies recognize a leukocyte differentiation antigen.

In some embodiments, the plurality of antibodies are anti-CD3 antibody, anti-CD4 antibody, anti-CD19 antibody, anti-CD16 antibody, or anti-CD56 antibody.

In some embodiments, the transistor sensor further comprises a microfluidic component. The microfluidic component has a microfluidic channel extending in a second direction different from the first direction to allow fluid containing animal cells to pass through the microfluidic channel, and the microfluidic component is disposed on the transistor sensor to allow the animal cells in the microfluidic channel to pass through the cell detection layer.

Other embodiments of the present disclosure provide a method of using a transistor sensor, including providing a cell sample; adding the cell sample to a transistor sensor, wherein the transistor sensor includes a field effect transistor, a surface modification layer, and a cell detection layer. The surface modification layer is disposed on the field effect transistor. The cell detection layer is disposed on the surface modification layer and contains a plurality of antibodies, wherein the antibodies are configured to identify a cell surface antigen, and the cell detection layer is configured to capture cells identified by the antibodies. The method of using the transistor sensor further includes detecting the electric signal from the field effect transistor of the transistor sensor.

In some embodiments, the cell sample is derived from a cell culture, a blood sample, or a tumor tissue sample.

In some embodiments, the cell surface antigen is a leukocyte differentiation antigen.

In some embodiments, the method of using the transistor sensor further includes adding a buffer solution to remove other cells not captured by the cell detection layer after adding the cell sample to the transistor sensor.

In some embodiments, the cells in the cell sample are immune cells.

### BRIEF DESCRIPTION OF THE DRAWINGS

Aspects of the present disclosure are best understood from the following detailed description when read with the accompanying Figures. It is noted that, in accordance with the standard practice in the industry, various features are not drawn to scale. In fact, the dimensions of the various features may be arbitrarily increased or reduced for clarity of discussion.

FIG. 1A illustrates a cross-sectional view of a transistor sensor according to some embodiments.

FIG. 1B illustrates a top view of the transistor sensor of FIG. 1A.

FIG. 1C illustrates a schematic cross-sectional view of the transistor sensor of FIG. 1A in operation.

FIG. 2 illustrates a schematic exploded view of a transistor sensor according to some embodiments.

FIG. 3 shows the electrical measurement results of human T cells detected by a transistor sensor provided with anti-CD3 antibody according to Experimental Example 1.

FIG. 4 shows the electrical measurement results of human T cells detected by a transistor sensor provided with anti-CD20 antibody according to Experimental Example 2.

FIG. 5 shows the electrical measurement results of human T cells and B cells detected by a transistor sensor provided with anti-CD3 antibody according to Experimental Example 3.

FIG. 6 shows the electrical measurement results of human T cells and B cells detected by a transistor sensor provided with anti-CD20 antibody according to Experimental Example 4.

FIG. 7 shows the electrical measurement results of detecting human B cells and different concentrations of T cells by a transistor sensor provided with anti-CD3 antibodies according to experimental example 5.

### DETAILED DESCRIPTION

In order to make the description of this disclosure more detailed and complete, the following is an illustrative description of the embodiments and specific examples of this disclosure; but, this is not the only way to practice or use the specific embodiments of the present disclosure. The embodiments disclosed below can be combined or replaced with each other under beneficial circumstances, and other embodiments can be added to an embodiment without further description or explanation.

In the following description, numerous specific details will be set forth in detail to enable readers to fully understand the following embodiments. However, embodiments of the present disclosure may be practiced without these specific details. In other cases, in order to simplify the drawings, well-known structures and devices are only schematically shown in the drawings.

In addition, the present disclosure may repeat reference numerals and/or letters in the various examples. This repetition is for the purpose of simplicity and clarity and does not in itself dictate a relationship between the various embodiments and/or configurations discussed. In addition, in the following disclosure, the formation of a first feature over or on a second feature in the description that follows may include embodiments in which the first and second features are formed in direct contact, and may also include embodiments in which additional features may be formed between the first and second features, such that the first and second features may not be in direct contact.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

A field effect transistor (FET) is a semiconductor component extremely sensitive to adjacent charges. When a field effect transistor of a sensor is exposed to an aqueous environment containing a target substance, such as protein, DNA, or RNA, the target substance binds to recognition molecules on the surface of the field effect transistor. In this case, the electric field formed by the charge carried by the target substance affects the number of electrons or holes in the semiconductor channel, triggering the increase or decrease of conductivity. By monitoring changes in the electrical conductivity, the presence of the target substance can be detected, and even its concentration can be measured.

Some embodiments disclosed herein provide a field effect transistor sensor to detect whether a specific type of cells is present in a cell sample. The principle of sensing is based on that after the antibodies capture cells, the charges on the cell membrane surfaces of the captured cells can be measured. In some embodiments, when the sensing surface of the transistor sensor carries negatively charged sensing targets (e.g., negatively charged antigens), it is necessary to increase the voltage before the drain current starts to rise. In other embodiments, when the sensing surface of the transistor sensor carries positively charged sensing targets (e.g., positively charged antigens), a lower voltage value is sufficient to observe an increase in drain current. The basis of electrical measurements relies on the difference in electrical signals between the reference electrical curve and the control group, primarily observed in the I-V scans along the V-axis. If the test sample contains the target cells, the detection curve will deviate from the control electrical curve obtained from the control solution, and the degree of deviation increases with higher concentrations of the target cells.

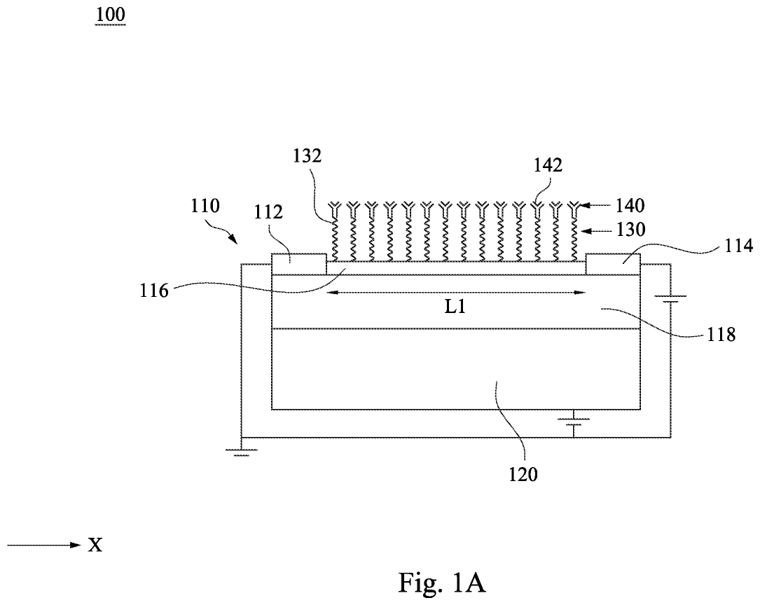

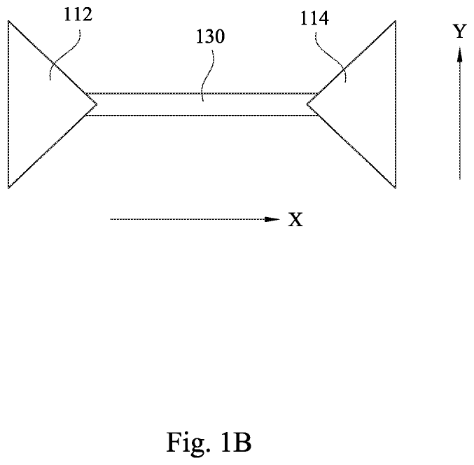

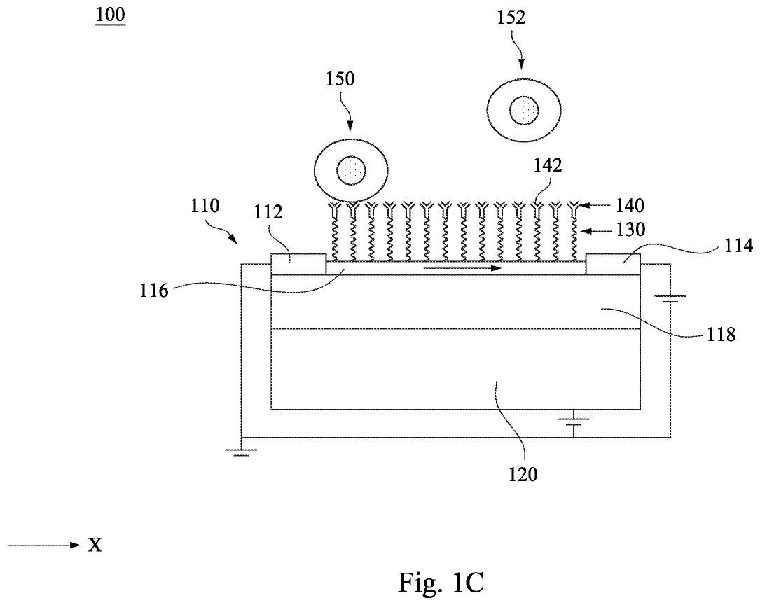

Referring to FIGS. 1A and 1B, which illustrate a cross-sectional view and a top view of a transistor sensor according to some embodiments. The transistor sensor 100 includes a field effect transistor 110, a surface modification layer 130, and a cell detection layer 140.

The field effect transistor 110 includes a source region 112, a drain region 114, a semiconductor channel 116, a gate dielectric layer 118, and a gate electrode 120. The source region 112 and the drain region 114 are spaced apart in the first direction (i.e., the X direction). The semiconductor channel 116 extends in the first direction and is disposed between the source region 112 and the drain region 114. The gate dielectric layer 118 is disposed under the semiconductor channel 116. The gate electrode 120 is disposed under the gate dielectric layer 118.

As shown in FIG. 1A, a surface modification layer 130 is disposed on the semiconductor channel 116. The cell detection layer 140 is disposed on the surface modification layer 130. The cell detection layer 140 includes a plurality of antibodies 142. The plurality of antibodies 142 are used to identify cell surface antigens. The cell detection layer 140 is configured to capture cells recognized by the antibody. The antibodies 142 may include, but are not limited to, monoclonal antibodies, human antibodies, humanized antibodies, and chimeric antibodies.

As shown in FIGS. 1A and 1B, in some embodiments, the semiconductor channel 116 has a first length L1 in the first direction X to allow the cell detection layer 140 of the transistor sensor 100 to capture cells. In some embodiments, the first length L1 of the semiconductor channel 116 ranges from 50 micrometers (μm) to 1000 μm, for example, from 100 μm to 500 μm. In some embodiments, the semiconductor channel 116 also has a width in the second direction (i.e., the Y direction). In some embodiments, the width ranges from 50 μm to 1000 μm, such as 100 μm to 500 μm.

In some embodiments, the material of the semiconductor channel may be, for example, polycrystalline silicon or monocrystalline silicon. In some embodiments, the surface modification layer 130 is formed on the semiconductor channel 116 and includes a plurality of connection portions 132 formed away from the semiconductor channel 116.

In some specific examples, the surface modification layer 130 is formed by the following procedures. Specifically, the semiconductor channel 116 is subjected to oxygen plasma treatment to create hydroxyl groups on its surface, making the surface of the semiconductor channel 116 more hydrophilic. Then, the semiconductor channel 116 is immersed in a solution of 3-aminopropyl triethoxysilane (APTES) to form an amino-terminal monolayer on the surface of the semiconductor channel. Subsequently, the semiconductor channel 116 is immersed in a glutaraldehyde (GA) solution to create a surface modification layer 130 on which several terminal aldehyde groups (i.e., the connection portion 132) are disposed on the surface.

In one embodiment, after cleaning the surface of the semiconductor channel 116 with acetone and alcohol to remove impurities, the surface of the semiconductor channel 116 is treated with 18-watt (W) oxygen plasma for 60 seconds. Next, the transistor sensor 100 is immersed in a 2% APTES (3-aminopropyltriethoxysilane) alcohol solution and shaken for 30 minutes. After rinsing with alcohol, the transistor sensor 100 is heated to 120° C. for 10 minutes to remove excess alcohol. Next, the transistor sensor 100 is soaked in a 2.5% glutaraldehyde Bis-Tris propane buffer solution and shaken for one hour, and then the solution is washed off. Finally, antibodies are added to react on the semiconductor channel 116 for one night, and then the unreacted aldehyde groups were blocked with 4 mM sodium cyanoborohydride.

The cell detection layer 140 binds to the surface modification layer 130 and can recognize and capture cells. Specifically, the plurality of antibodies 142 of the cell detection layer 140 are respectively bound to the plurality of connection portions 132 of the surface modification layer 130. In some embodiments, the formation of the semiconductor channel 116 having the surface modification layer 130 involves immersing the semiconductor channel 116 in a solution containing antibodies. This operation allows the amino groups in the antibodies to attach to the terminal aldehyde groups originally from the glutaraldehyde solution, thereby anchoring the antibodies to the surface of the surface modification layer 130.

Referring to FIG. 1C, a schematic diagram of the transistor sensor 100 in operation is shown. The target cell 150 recognized by the antibodies 142 of the cell detection layer 140 is captured and retained in the cell detection layer 140. Non-target cells 152 that are not recognized by the antibody 142 of the cell detection layer 140 are not captured in the cell detection layer 140. In some embodiments, non-target cells 152 flow out of the cell detection layer 140 on the semiconductor channel 116 of the transistor sensor 100 along with the buffer solution.

When the cell detection layer 140 captures the target cells 150, the field effect transistor 110 can detect the target cells 150 and a change in electrical properties occurs because the cell membrane surface of the target cells 150 is negatively charged and is close to the semiconductor channel 116.

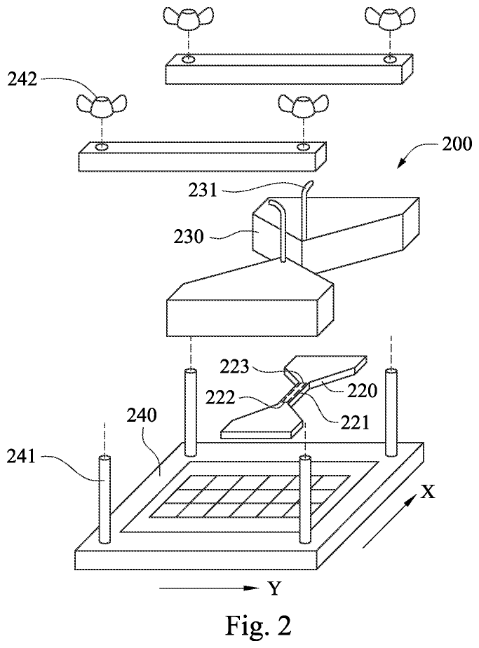

Referring to FIG. 2, a schematic exploded view of a transistor sensor including a fluid channel is shown. The transistor sensor 200 includes a microfluidic component 220 and an upper cover 230 covering the microfluidic component 220.

As shown in FIG. 2, a microfluidic channel 221 extending in the first direction (X direction) is defined in the microfluidic component 220 to allow a fluid containing cells to pass therethrough. The microfluidic component 220 can be, for example, made by molding polydimethylsiloxane (PDMS). The microfluidic channel 221 has an upstream end and a downstream end. The microfluidic component 220 is formed with an inlet 222 and an outlet 223, positioned respectively at the upstream and downstream ends of the microfluidic channel 221, for the mutual flow of fluid within the microfluidic channel 221.

The upper cover 230 is provided with two pipelines 231 connected to an injection pump (not shown). The upper cover 230 may be a transparent material and may be made of acrylic acid, for example. The pipelines 231 are aligned with the inlet 222 and the outlet 223 respectively.

The transistor sensor 200 can be clamped in a position on a metal platform 240 using a metal rod 241 and a nut 242.

When the transistor sensor 200 is used to detect cells in the sample, a syringe pump is used to fill the buffer for a period of time, so that the buffer solution flows into one of the pipelines 231, flows through the inlet 222, the microfluidic channel 221 and the outlet 223, and flows out from the other of the pipelines 231, so as to stabilize the transistor sensor 200 mainly composed of the field-effect transistor before measuring the ID-VG response.

In some embodiments, the transistor sensor 200, primarily based on the field effect transistor, is considered stable only after obtaining three consecutive overlapping drain current index-gate voltage curves (ID-VG curves). The final ID-VG curve is then used as a baseline for the subsequent bio-sensing process. Then, the buffer solution is removed from the microfluidic channel 221, and a test cell sample is filled for a period using the injection pump. Next, the injection pump is used to pump buffer solution into the microfluidic channel 221 for a period to remove any non-specific binding, followed by measuring the ID-VG response of the cell sample. In some embodiments, three consecutive overlapping ID-VG curves are required before a detection curve can be confirmed as the signal for the cell sample.

Because a semiconductor field effect sensing element only detects changes in electrical signals, the detection results can be easily disrupted by cells or impurities having charges in the sample. Therefore, the specificity of recognizing biological molecules for the captured target substance is highly crucial. Additionally, during the measurement process, it is also important to prevent interfering substances having the same charge from directly adhering to the surface of the sensor element, to avoid influencing the measurement results. In some embodiments, measurements are conducted in a closed, dry, and light-restricted environment during testing to reduce experimental errors.

The following describes test results from some experimental examples using transistor sensor 100 for detecting human lymphocytes in the sample, testing the sensitivity and specificity of transistor sensor 100.

Before a cell sample is detected using transistor sensor 100, a buffer solution is filled for a period using an injection pump to allow the buffer solution to flow through the cell detection layer 140 over the semiconductor channel 116. The field effect transistor's drain current index-gate voltage curve (ID-VG curve) is measured as the baseline for later detection of the cell sample.

Afterward, during the detection, the test cell sample is filled for a period using the injection pump, and the buffer solution is then removed from the microfluidic channel. Next, the injection pump is used to pump buffer solution into the microfluidic channel for a period to remove any non-specific binding, followed by measuring the ID-VG curve of the cell sample.

The cell concentration in the cell sample can be determined based on the signal difference between the ID-VG curve used as the baseline and the ID-VG curve obtained from measuring the cell sample. For example, this measurement can be done by comparing the threshold voltage of the ID-VG curve used as the baseline with the threshold voltage of the ID-VG curve obtained from measuring the cell sample.

### Experimental Example 1

T cells cultured from a human T cell line were tested, and the antibody 142 in the cell detection layer 140 of the transistor sensor 100 was anti-CD3 antibody. In the test, the concentration of T cells in the solution is 1.0×10⁵ cells/100 μL.

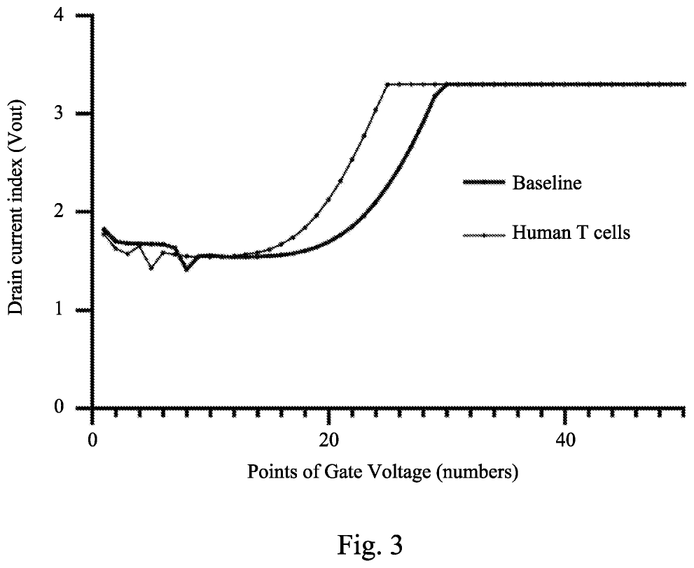

FIG. 3 shows the electrical measurement results of human T cells detected using the transistor sensor provided with anti-CD3 antibodies according to Experimental Example 1. The horizontal axis represents the points of the gate voltage (number), and the vertical axis represents the drain current index. It can be observed that the ID-VG curve shows a noticeable shift. Since human T cells express CD3, when anti-CD3 antibodies were set in the cell detection layer 140 of the transistor sensor 100, the field effect transistor 110 can detect human T cells captured by the anti-CD3 antibodies.

### Experimental Example 2

T cells cultured from a human T cell line were tested, and the antibody 142 in the cell detection layer 140 of the transistor sensor 100 was anti-CD20 antibody.

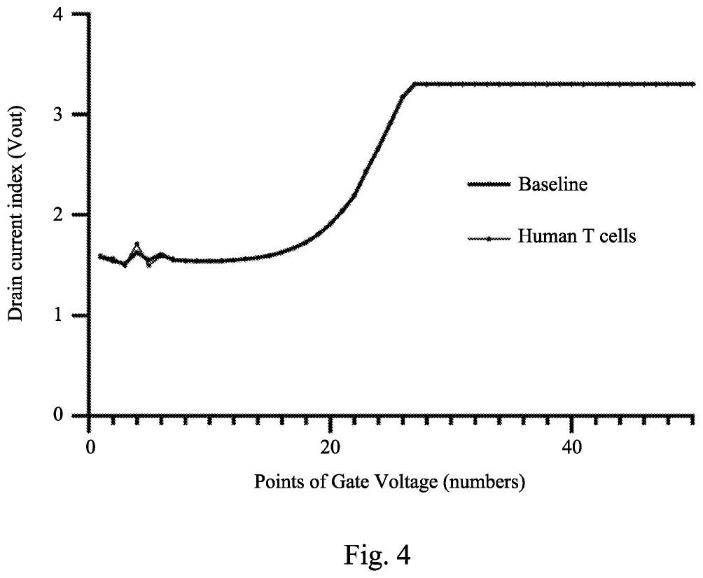

FIG. 4 shows the electrical measurements of human T cells using a transistor sensor provided with anti-CD20 antibodies according to Experimental Example 2. It can be seen that the ID-VG curve shows minimal to no shift. Since human T cells do not express CD20, when the transistor sensor was provided with anti-CD20 antibodies, it cannot recognize and capture human T cells. As a result, there is no significant change in the electrical characteristics of the field effect transistor 110.

### Experimental Example 3

T cells cultured from a human T cell line (i.e., the test group) and B cells cultured from a B cell line (i.e., the control group) were detected, and the antibody 142 in the cell detection layer 140 of the transistor sensor 100 was anti-CD3 antibody. The testing process included introducing the T cells into the microfluidic channel of the transistor sensor, followed by a 30-minute incubation period, during which the electrical signals of the transistor sensor were measured. Subsequently, the microfluidic channel was cleaned to remove any remaining cells, and a fresh buffer solution was added. Then, the B cells were introduced into the microfluidic channel of the transistor sensor, and after another 30-minute incubation period, the electrical signals of the transistor sensor were measured again.

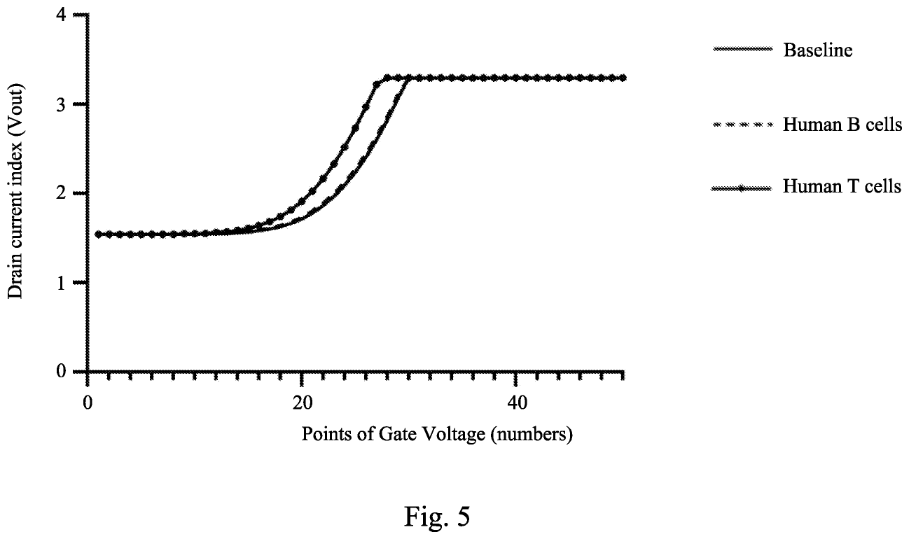

FIG. 5 shows the electrical measurement results of human T cells and B cells using the transistor sensor provided with anti-CD3 antibodies, according to Experimental Experiment 3. It can be observed that when the human T cells were tested, there was a significant shift in the ID-VG curve of field effect transistor 110. However, when the human B cells were tested, there was a slight shift in the ID-VG curve of field effect transistor 110. Therefore, the transistor sensor can clearly distinguish between target cells (e.g., T cells) and non-target cells (e.g., B cells).

### Experimental Example 4

B cells cultured from a human B cell line (i.e., the test group) and T cells cultured from a human T cell line (i.e., the control group) were detected, and the antibody 142 in the cell detection layer 140 of the transistor sensor 100 was anti-CD20 antibody. The testing process included introducing the T cells into the microfluidic channel of the transistor sensor, followed by a 30-minute incubation period, and the electrical signals of the transistor sensor were measured. Subsequently, the microfluidic channel was rinsed to remove residual cells, and a fresh buffer solution was added. Then, B cells were introduced into the microfluidic channel of the field effect transistor sensor and incubated for 30 minutes before measuring the electrical signals of the field effect transistor sensor again.

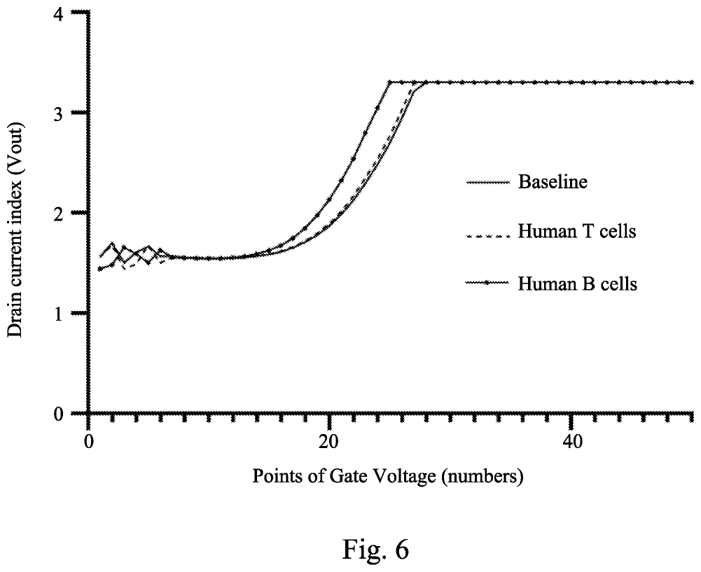

FIG. 6 shows the electrical measurement results of human B cells and T cells detected using the field effect transistor sensor provided with anti-CD20 antibodies, according to Experimental Example 4. It can be seen that the ID-VG curve of the field effect transistor 110 had an apparent deviation when the human B cells were tested. When the human T cells were tested, the ID-VG curve of field effect transistor 110 was slightly shifted. Therefore, the transistor sensor can clearly distinguish between target cells (e.g., B cells) and non-target cells (e.g., T cells).

Based on FIGS. 5 and 6, it is evident that the electrical changes in target cells and non-target cells can be clearly distinguished. Therefore, the transistor sensor 100 can specifically identify target cells in the sample.

### Experimental Example 5

B cells cultured from a human B cell line (i.e., the control group) and T cells cultured from a human T cell line (i.e., the test group) were detected, and the antibody 142 in the cell detection layer 140 of the transistor sensor 100 was anti-CD3 antibody. The concentration of human B cells detected was 10×10⁵ cells/100 μL. The concentrations of human T cells tested in the sample were 1.0×10³ cells/100 μL, 1.0×10⁴ cells/100 μL, and 1.0×10⁵ cells/100 μL, respectively. The testing process included testing the B cells after establishing the baseline, rinsing the sensor to remove B cells, and subsequently testing the T cell samples of different concentrations in sequence. Between the different T cell samples of different concentrations, the sensor was washed with buffer solution and the cells in the previous sample were removed.

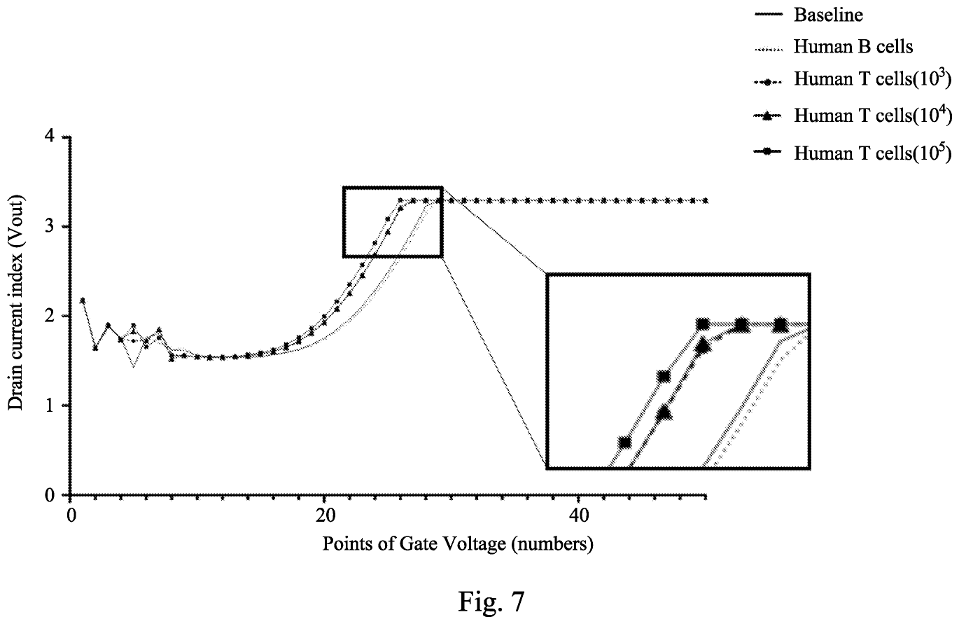

FIG. 7 shows the electrical measurement results of testing human B cells and different concentrations of T cells using the transistor sensor provided with anti-CD3 antibodies, according to Experimental Example 5. It shows that when a sample of B cells was tested, the obtained electrical curve was close to the baseline. When the samples contained 1.0×10³ cells/100 μL, 1.0×10⁴ cells/100 μL, and 1.0×10⁵ cells/100 μL T cells, it can be seen that the electrical curves were obviously shifted.

Some embodiments of the present disclosure provide a method of using a transistor sensor, including providing a cell sample; adding the cell sample to a transistor sensor including a field effect transistor; and detecting the electric signals of the field effect transistor of the transistor sensor. The sensor includes a field effect transistor, a surface modification layer and a cell detection layer. The surface modification layer is disposed on the field effect transistor. The cell detection layer is disposed on the surface modification layer and contains a plurality of antibodies, and these antibodies are configured to recognize cell surface antigens, and the cell detection layer is configured to capture cells recognized by these antibodies.

The transistor sensor provided by this disclosure can be applied to detect samples from cell culture, blood samples, or tumor tissue samples, or the like. In some embodiments, the antibody 142 in the cell detection layer 140 of the transistor sensor 100 can recognize a leukocyte differentiation antigen, so the transistor sensor 100 can be used to detect different types of leukocytes.

In the conventional way, certain pretreatment is required before detecting immune cells in the tumor. Without the pretreatment, the tumor tissue contains many kinds of cells. In the past, cells from the tumor were processed to determine the type of each cell and facilitate subsequent experiments. This involves actions such as single-cell sorting and isolation to obtain the desired cells; however, the process is cumbersome and requires more resources and time. The transistor sensor in the embodiments of the disclosure can be used and achieve good results in the pretreatment. The transistor is specific, sensitive, and fast, which can achieve better sorting and reduce a lot of unnecessary expenses and time.

Some embodiments of the present disclosure provide a method for detecting immune cell types, such as detecting lymphocyte subtypes in cell samples. Lymphocyte subtypes may be, for example, T cells, B cells, NK cells, CD4 T cells, CD8 T cells, or the like. The cell sample may be, for example, a blood sample, a cell sample separated from blood, a cell sample from a tumor, a cultured cell sample, or the like. Different subtypes of lymphocytes respectively have specific surface markers of lymphocytes, for example, T cells have CD3 surface marker, B cells have CD19 surface marker, NK cells have CD16 surface marker and CD56 surface marker, CD4 T cells have CD4 surface marker, and CD8 T cells have CD8 surface marker. In some embodiments, the cell detection layers of multiple transistor sensors are respectively provided with different antibodies that recognize different cell surface markers. For example, the first transistor sensor is provided with anti-CD3 antibodies, the second transistor sensor is provided with anti-CD19 antibodies, the third transistor sensor is provided with anti-CD16 antibodies and anti-CD56 antibodies, the fourth transistor sensor is provided with anti-CD4 antibodies, and the fifth transistor sensor is provided with anti-CD8 antibodies. Take the solution containing the cell sample, for example, take 1 to 10 μL of the solution, and separately add the aliquots to multiple transistor sensors respectively provided with different antibodies, such as the aforementioned first to fifth transistor sensors. Based on the presence or absence of electrical signals detected by each transistor sensor targeting different cell types and the magnitude of these signals, the presence or absence, as well as the relative proportion of the specific lymphocyte subpopulations to be detected in the sample, can be determined.

In some embodiments, relative detection curves for cell numbers can be established by measuring the electrical signals detected from different quantities of cells. Subsequently, the number of cells detected in the sample can be determined by extrapolating from the magnitude of the electrical signals.

The transistor sensor provided by the various embodiments of the present disclosure can quickly detect cell types. In other words, by adding the sample to the field effect transistor sensor equipped with specific antibodies, it is possible to immediately determine whether the cells of interest (such as specific lymphocyte subtypes) are present in the sample and/or their proportion in the sample.

Currently, the mainstream of cell detection technology for clinical samples is to analyze cells by flow cytometry or by optical signal interpretation. There are many complex pre-processing steps for the samples, leading to decreased cell viability and susceptibility of genes to damage. After the pre-processing steps, the detection time for the instrument is long, and the consumables for the detection are costly. Further, residual substances in the instrument's tubing can affect the results. In comparison, the transistor sensing elements of the embodiments of the present disclosure have the advantages of rapidity, label-free, low cost, and high sensitivity in detecting the corresponding biomarkers on the cell surface.

Immunotherapy is a hot research field in cancer treatment, which can change the specificity and selectivity of T lymphocytes by inducing relevant cytotoxic T lymphocytes to transfer to patients after they are vaccinated. Immune cells can distinguish specific antigens of tumor cells or self-antigens from countless other antigens with high sensitivity and selectivity. However, in the past, it took a lot of time and money to test whether antigens were related to cells, such as the analysis technology of flow cytometry. The transistor sensor of the present disclosure uses semiconductor field effect transistor components to detect cells, taking advantage of its speed, immediacy, and high specificity for sensing. This transistor sensor can also be manufactured as a portable sensor, providing greater convenience. Furthermore, by utilizing transistor sensors containing field effect transistors and specific antibodies, the cells in the sample can be detected without labeling the cells in advance, so a lot of reagents and consumables can be saved, and the test results can be obtained quickly.

Although the present disclosure has been disclosed in many embodiments and examples, it is not intended to limit the present disclosure. Anyone skilled in the art can make various changes and modifications without departing from the spirit and scope of the present disclosure. Therefore, the scope of protection of the present disclosure shall be subject to the scope of the appended claims.

What is claimed is:

1. A transistor sensor, comprising:
a field effect transistor including:
a source region;
a drain region spaced apart from the source region in a first direction;
a semiconductor channel extending in the first direction and disposed between the source region and the drain region;
a gate dielectric layer disposed under the semiconductor channel; and
a gate disposed under the gate dielectric layer;
a surface modification layer disposed on the semiconductor channel; and
a cell detection layer disposed on the surface modification layer and containing a plurality of antibodies, wherein the antibodies are configured to identify a cell surface antigen, and the cell detection layer is configured to capture a cell identified by the antibodies.

2. The transistor sensor of claim 1, wherein the cell surface antigen is a leukocyte differentiation antigen.

3. The transistor sensor of claim 1, wherein the semiconductor channel has a length in the first direction to allow the cell detection layer to capture the cell.

4. The transistor sensor of claim 3, wherein the length is in a range from about 50 μm to 1000 μm.

5. The transistor sensor of claim 1, wherein the surface modification layer comprises siloxane compounds.

6. The transistor sensor of claim 1, wherein the surface modification layer is connected with the plurality of antibodies of the cell detection layer through a plurality of terminal aldehyde groups.

7. The transistor sensor of claim 1, wherein the cell is a white blood cell.

8. The transistor sensor of claim 1, wherein the plurality of antibodies recognize a leukocyte differentiation antigen.

9. The transistor sensor of claim 1, wherein the plurality of antibodies are anti-CD3 antibody, anti-CD4 antibody, anti-CD19 antibody, anti-CD16 antibody, or anti-CD56 antibody.

10. The transistor sensor according to claim 1, further comprising:
a microfluidic component having a microfluidic channel extending in a second direction different from the first direction to allow fluid containing the cell to pass through the microfluidic channel, and the microfluidic component is disposed on the transistor sensor to allow the cell in the microfluidic channel to pass through the cell detection layer.

11. A method for using a transistor sensor, comprising:
providing a cell sample;
adding the cell sample to a field effect transistor sensor, wherein the field effect transistor sensor comprises:
a field effect transistor;
a surface modification layer disposed on the field effect transistor; and
a cell detection layer disposed on the surface modification layer and containing a plurality of antibodies, wherein the antibodies are configured to identify a cell surface antigen, and the cell detection layer is configured to capture a cell identified by the antibodies; and
detecting electric signals of the field effect transistor of the transistor sensor.

12. The method for using the transistor sensor of claim 11, wherein the cell sample is from cell culture, blood sample, or tumor tissue sample.

13. The method for using the transistor sensor of 11, wherein the cell surface antigen is a leukocyte differentiation antigen.

14. The method for using the transistor sensor of claim 11, further comprising: adding a buffer solution to remove other cells not captured by the cell detection layer after adding the cell sample to the transistor sensor.

15. The method for using the transistor sensor of claim 11, wherein the cell is an immune cell.

16. The method for using the transistor sensor of claim 11, wherein the plurality of antibodies are anti-CD3 antibody, anti-CD4 antibody, anti-CD19 antibody, anti-CD16 antibody, or anti-CD56 antibody.
